(19) United States
(12) Patent Application Publication
(10) Pub. No.: US 2026/0110672 A1
(43) Pub. Date: Apr. 23, 2026

(54) ILLUMINATED SMOKE DETECTOR ASSEMBLY

(71) Applicant: Jose Sandoval, Cicero, IL (US)

(72) Inventor: Jose Sandoval, Cicero, IL (US)

(21) Appl. No.: 18/923,172

(22) Filed: Oct. 22, 2024

(51) Int. Cl. G01N 33/00; F21V 33/00; F21Y 105/18; F21Y 113/00; F21Y 115/10

(52) U.S. Cl. CPC G01N 33/0027; F21V 33/0076; F21Y 2105/18; F21Y 2113/00; F21Y 2115/10

(57) ABSTRACT

An illuminated smoke detector assembly includes a housing, a light cover, a plurality of LED lights, a smoke detector unit, a cover plate, and a plurality of apertures. The light cover and the housing together are designed to define a ring chamber positioned between an inner side wall of the light cover and an outer side wall of the housing. The LED lights are positioned within the ring chamber. The smoke detector unit is surrounded by the inner side wall of light cover. The cover plate is attached to the smoke detector unit to obscure the smoke detector unit from view. The apertures are positioned in each of the outer side wall of the housing and the inner side wall of the light cover. The apertures are designed to permit passage of smoke through the housing and the light cover to the smoke detector unit.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to smoke detectors and more particularly pertains to a new illuminated smoke detector assembly for providing a smoke detector that is hidden from view within a light fixture. Smoke detectors can be unsightly, so concealing them within light fixtures could improve aesthetics. However, the smoke detector must still be able to function by receiving smoke or other airborne contaminants, such as carbon monoxide. The illuminated smoke detector assembly includes apertures for the passage of air and airborne contaminants to the smoke detector unit inside the light fixture.

### (2) Description of Related Art Including Information Disclosed under 37 CFR 1.97 and 1.98

The prior art relates to smoke detectors, including smoke detectors that can be used in conjunction with light fixtures. The prior art, as best understood, does not disclose an illuminated smoke detector assembly that includes a housing, a light cover, a plurality of LED lights, a smoke detector unit, a cover plate, and a plurality of apertures, wherein the light cover and the housing together are designed to define a ring chamber with the LED lights in the ring chamber, which ring chamber surrounds the smoke detector unit. The cover plate is attached to the smoke detector unit to obscure the smoke detector unit from view. The apertures are positioned in each of an outer side wall of the housing and an inner side wall of the light cover to permit passage of smoke through the housing and the light cover to the smoke detector unit.

### BRIEF SUMMARY OF THE INVENTION

An embodiment of the disclosure meets the needs presented above in a illuminated smoke detector assembly generally comprising a housing, a light cover, a plurality of LED lights, a smoke detector unit, a cover plate, and a plurality of apertures. The housing includes a top panel and an outer side wall. The top panel is disk-shaped and includes a perimeter panel edge. The outer side wall is cylindrical. The outer side wall extending perpendicularly from the perimeter panel edge. The outer side wall includes a lip edge. The light cover is detachably connected to the housing. The light cover includes a ring-shaped wall portion extending perpendicularly from the outer side wall at the lip edge of the outer side wall. The light cover includes an inner side wall extending transversely from the ring-shaped wall portion to the top panel of the housing. The light cover and the housing together are designed to define a ring chamber positioned between the inner side wall and the outer side wall. The light cover includes translucent material to permit passage of light through the light cover. The LED lights are attached to the housing. The LED lights are positioned within the ring chamber. The smoke detector unit is detachably mounted on the top panel of the housing. The smoke detector unit is surrounded by the inner side wall of light cover. The cover plate is attached to the smoke detector unit. The cover plate is designed to extend outwardly toward and parallel to the ring-shaped wall of the light cover to obscure the smoke detector unit from view. The apertures are positioned in each of the outer side wall of the housing and the inner side wall of the light cover. The apertures are designed to permit passage of smoke through the housing and the light cover to the smoke detector unit.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an illuminated smoke detector assembly according to a first embodiment of the disclosure.

FIG. 2 is an exploded view of the first embodiment of the disclosure.

FIG. 3 is a top view of the first embodiment of the disclosure.

FIG. 4 is a front view of the first embodiment of the disclosure.

FIG. 5 is a cross-sectional view of the first embodiment of the disclosure.

FIG. 6 is a perspective view of the first embodiment of the disclosure in use.

FIG. 7 is a perspective view of a second embodiment of the disclosure.

FIG. 8 is a cross-sectional view of the second embodiment of the disclosure.

FIG. 9 is a front view of a third embodiment of the disclosure.

### DETAILED DESCRIPTION OF THE INVENTION

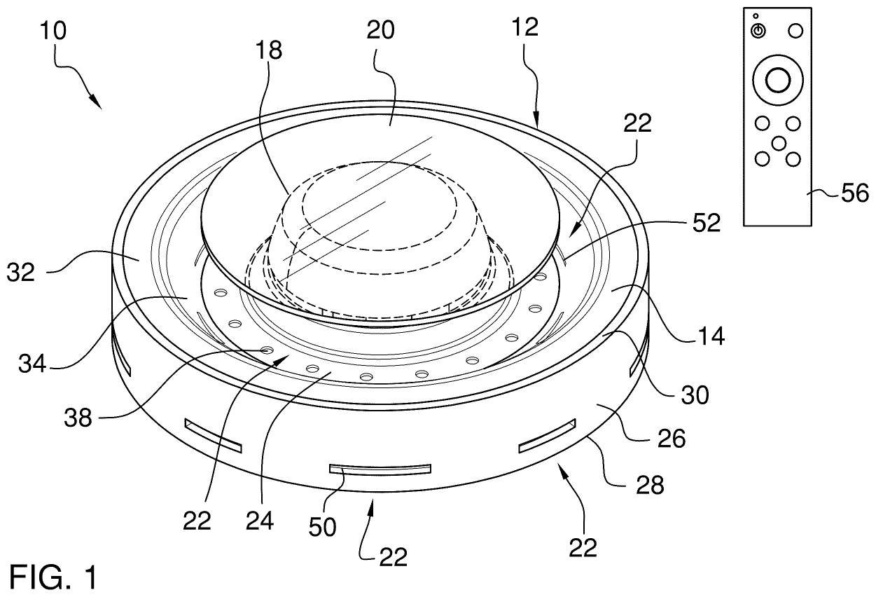

With reference now to the drawings, and in particular to FIGS. 1 through 9 thereof, a new illuminated smoke detector assembly embodying the principles and concepts of an embodiment of the disclosure and generally designated by the reference numeral 10 will be described.

In the first embodiment shown in FIGS. 1 through 6, an illuminated smoke detector assembly 10 includes a housing 12, a light cover 14, a plurality of LED lights 16, a smoke detector unit 18, a cover plate 20, and a plurality of apertures 22. The housing 12 includes a top panel 24 and an outer side wall 26. The top panel 24 is disk-shaped and includes a perimeter panel edge 28. The outer side wall 26 is cylindrical. The outer side wall 26 extends perpendicularly from the perimeter panel edge 28. The outer side wall 26 includes a lip edge 30. The light cover 14 is detachably connected to the housing 12. The light cover 14 includes a ring-shaped wall portion 32 extending perpendicularly from the outer side wall 26 at the lip edge 30 of the outer side wall 26. The light cover 14 includes an inner side wall 34 extending transversely from the ring-shaped wall portion 32 to the top panel 24 of the housing 12. The light cover 14 and the housing 12 together are designed to define a ring chamber 36 positioned between the inner side wall 34 and the outer side wall 26. The light cover 14 includes translucent material to permit passage of light through the light cover 14. The LED lights 16 are attached to the housing 12. The LED lights 16 are positioned within the ring chamber 36. The smoke detector unit 18 is detachably mounted on the top panel 24 of the housing 12. The smoke detector unit 18 is surrounded by the inner side wall 34 of light cover 14. The cover plate 20 is attached to the smoke detector unit 18. The cover plate 20 is designed to extend outwardly toward and parallel to the ring-shaped wall portion 32 of the light cover 14 to obscure the smoke detector unit 18 from view. The apertures 22 are positioned in each of the outer side wall 26 of the housing 12 and the inner side wall 34 of the light cover 14. The apertures 22 are designed to permit passage of smoke through the housing 12 and the light cover 14 to the smoke detector unit 18.

The apertures 22 include top panel apertures 38 positioned in the top panel 24 of the housing 12.

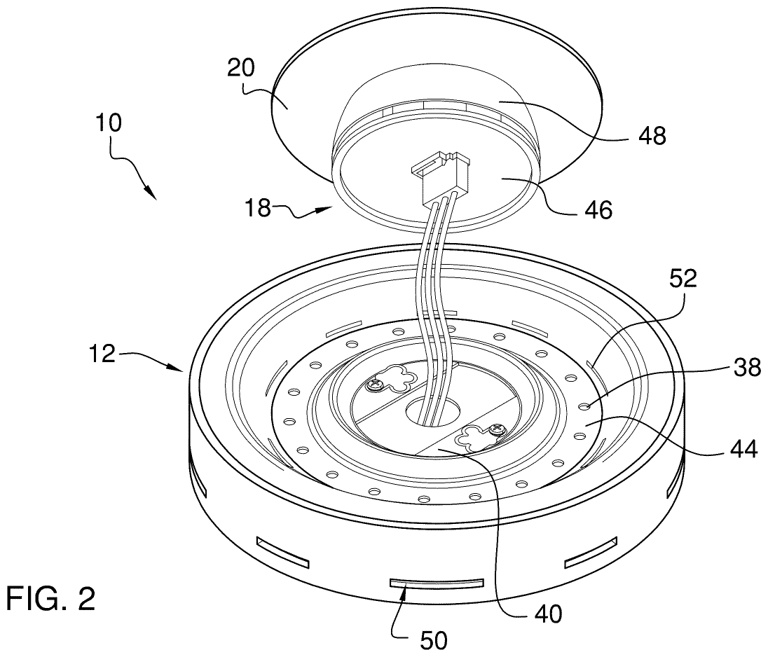

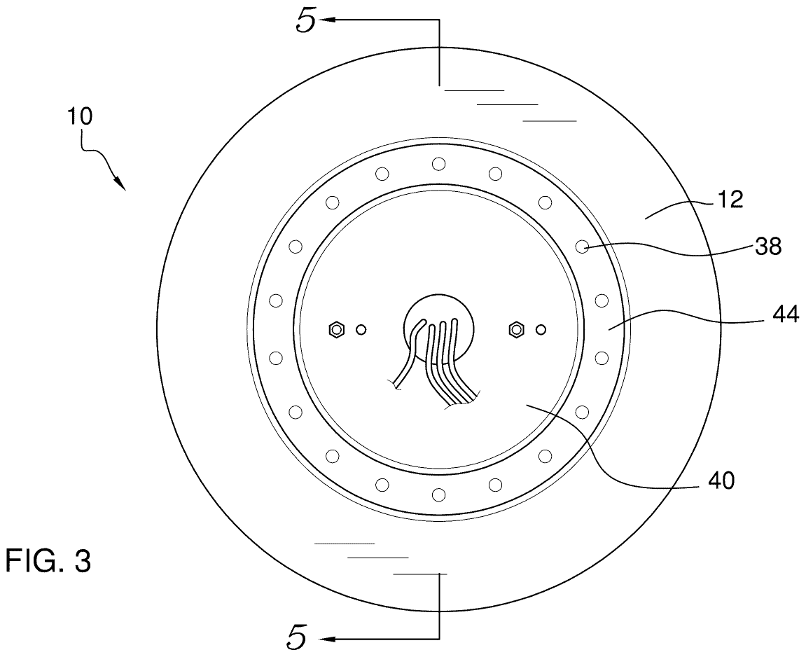

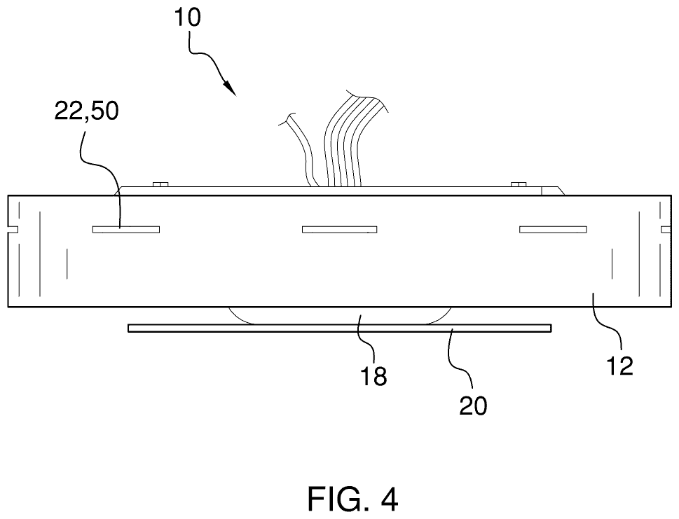

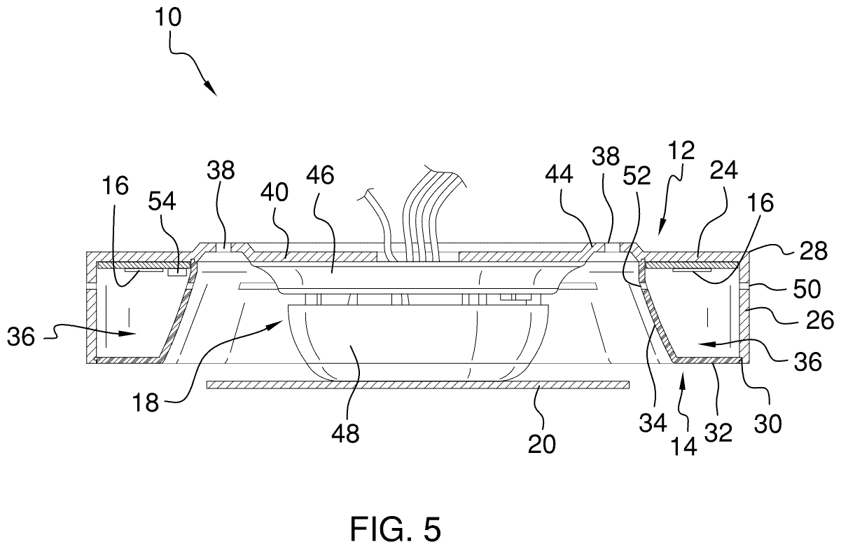

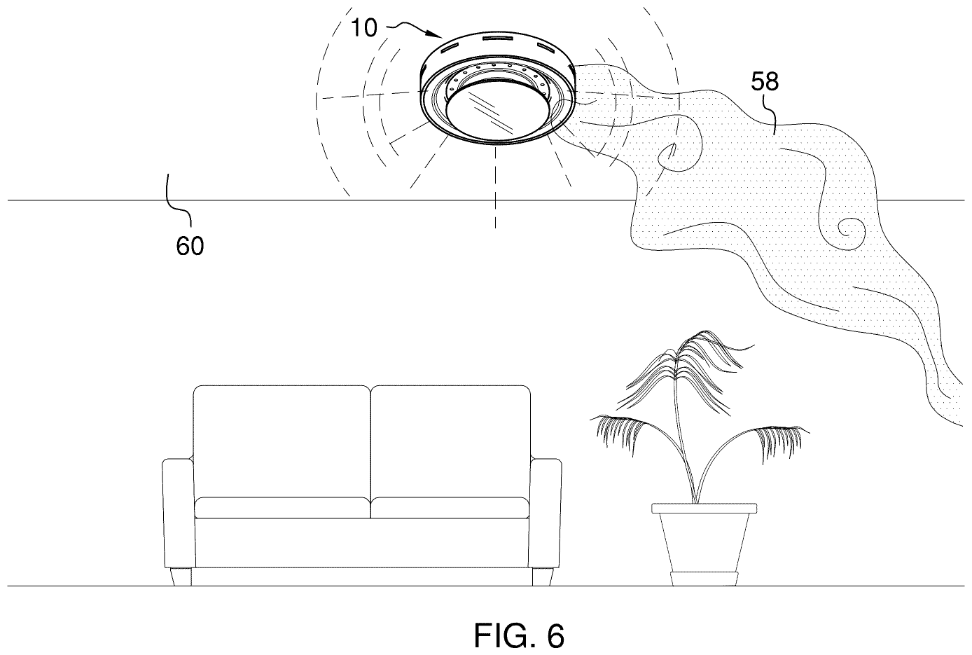

As shown in FIGS. 2 and 3, the top panel 24 includes a central mounting portion 40 designed to be attached to a surface, such as a ceiling 42 as shown in FIG. 6. The top panel 24 includes a perimeter portion 44 about the central mounting portion 40. The top panel apertures 38 are positioned in the perimeter portion 44. As best seen in FIGS. 4 and 5, the central mounting portion 40 is raised with respect to the perimeter portion 44 such that there is space between the perimeter portion 44 and the surface to which the central mounting portion 40 is attached to permit flow of smoke through the space to the top panel apertures 38. The top panel apertures 38 are positioned in a ring about the central mounting portion 40 between the smoke detector unit 18 and the inner side wall 34 of the light cover 14.

The smoke detector unit 18 includes a base portion 46 detachably connected to the top panel 24 of the housing 12 and a detector portion 48 detachably connected to the base portion 46. The cover plate 20 is attached to the detector portion 48 of the smoke detector unit 18.

As shown in FIG. 1, the apertures 22 include outer side wall apertures 50 positioned in the outer side wall 26 of the housing 12. The apertures 22 include inner side wall apertures 52 positioned in the inner side wall 34 of the light cover 14.

Each of the housing 12 and the cover plate 20 includes opaque material designed to block passage of light. In one possible embodiment, the smoke detector unit 18 is designed to also detect carbon monoxide.

In the exemplary embodiment shown in FIG. 1, the illuminated smoke detector assembly 10 further includes a control unit 54 that is connected to the LED lights 16 to control operation thereof. The control unit 54 is a wireless receiver positioned in the ring chamber 36, and the illuminated smoke detector assembly 10 further includes a remote control 56 designed to communicate with the control unit 54 to control operation of the LED lights 16, such as turning them on and off or increasing and decreasing the brightness.

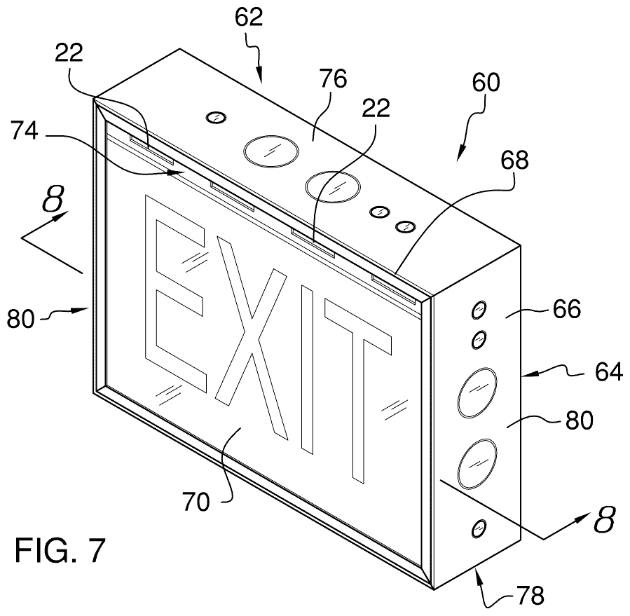

As shown in FIGS. 6, 7he smoke detector unit 18 is concealed within the housing 12 to preserve aesthetics, but the housing 12 has apertures 22 to permit the smoke detector to still receive airborne contaminants, such as smoke 58. Installation is simplified because a common opening can be used for the wiring of the light fixture and the wiring of the smoke detector unit 18. The other embodiments discussed herein also operate in a similar manner, though the designs of their housings and light fixtures are different.

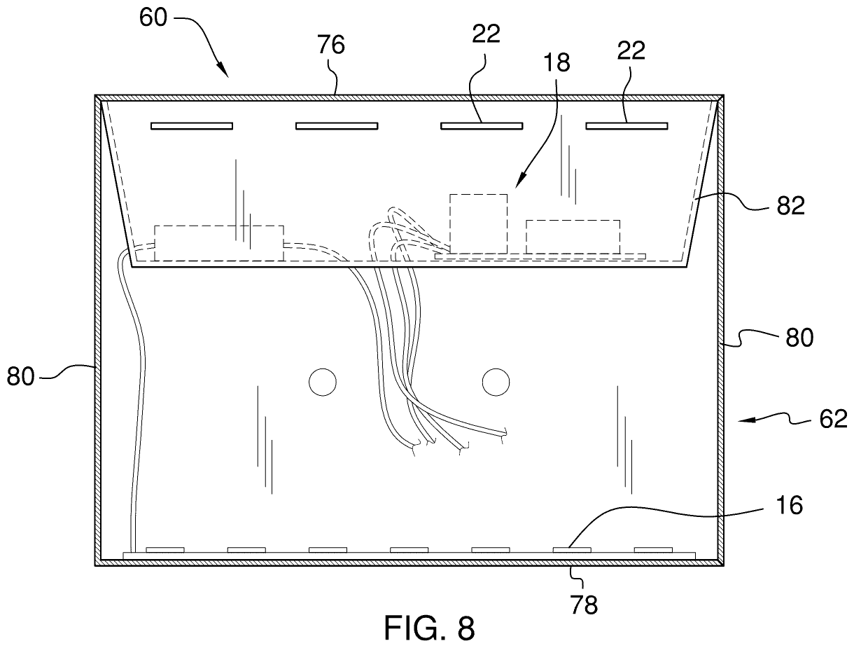

With reference to the second embodiment shown in FIGS. 7 and 8, the illuminated smoke detector assembly 60 has a box-shaped design and can be used for signage, such as an EXIT sign as shown. In this embodiment, the housing 62 includes a rear panel 64 and a perimeter wall 66 extending perpendicularly from the rear panel 64. The perimeter wall 66 includes a lip edge 68 defining an opening in the housing 62. A light cover 70 is detachably connected to the housing 62. The light cover 70 is a panel extending perpendicularly from the perimeter wall 66 at the lip edge 68 of the perimeter wall 66. The light cover 70 and the housing 62 together are designed to define a rectangular chamber 72. The light cover 70 is smaller than the opening in the housing 62 to create a gap 74 between the light cover 70 and the lip edge 68 to permit passage of smoke into the rectangular chamber 72. The light cover 70 includes translucent material to permit passage of light through the light cover 70. The plurality of LED lights 16 is attached to the housing 62. The LED lights 16 are positioned within the rectangular chamber 72. The smoke detector unit 18 is detachably mounted to the housing 62 within the rectangular chamber 72. The perimeter wall 66 includes a top wall portion 76, a bottom wall portion 78, and two lateral wall portions 80 connecting the top wall portion 76 and the bottom wall portion 78. The smoke detector unit 18 includes a cover frame 82 designed to house components of the smoke detector unit 18. The cover frame 82 is attached to the top wall portion 76.

The gap 74 is positioned between the light cover 70 and the lip edge 68 at the top wall portion 76. The cover frame 82 is designed and positioned to block the gap 74. The cover frame 82 includes a plurality of apertures 22 adjacent the gap 74 to permit passage of smoke through the cover frame 82. The light cover 70 can include instructional information to assist in exiting an area of a building in an emergency.

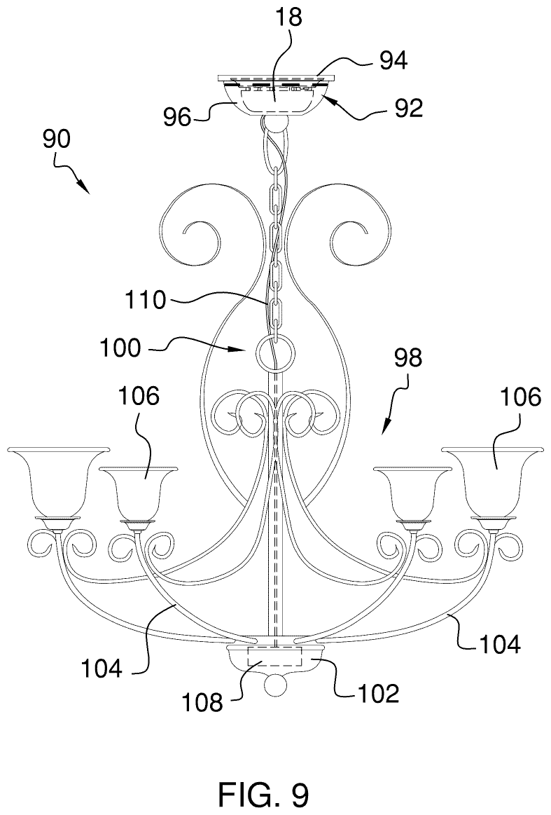

With reference to the third embodiment shown in FIG. 9, the illuminated smoke detector assembly 90 includes a housing 92 that includes a base plate 94 and a cover 96 detachably connected to the base plate 94. The cover 96 is dome-shaped. The base plate 94 is designed to be attached to a ceiling 42. The smoke detector unit 18 is detachably mounted on the base plate 94 within the cover 96. The plurality of apertures 22 is positioned in the cover 96 of the housing 92. The apertures 22 are designed to permit passage of smoke through the cover 96 to the smoke detector unit 18. A chandelier 98 is connected to and designed to be suspended from the cover 96 of the housing 92.

The chandelier 98 includes a central support assembly 100 connected to the cover 96, a hub 102 positioned at a free end of the central support assembly 100, a plurality of arms 104 extending from the hub 102, and a plurality of lighting fixtures 106 connected to the arms 104. The illuminated smoke detector assembly 90 further includes a battery 108 and wiring 110 connecting the battery 108 to the smoke detector unit 18. The battery 108 is positioned within the hub 102. The wiring 110 extends along the central support assembly 100 from the hub 102 to the housing 92.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An illuminated smoke detector assembly comprising:
a housing including a top panel and an outer side wall, said top panel being disk-shaped and including a perimeter panel edge, said outer side wall being cylindrical, said outer side wall extending perpendicularly from said perimeter panel edge, said outer side wall including a lip edge;
a light cover detachably connected to said housing, said light cover including a ring-shaped wall portion extending perpendicularly from said outer side wall at said lip edge of said outer side wall, said light cover including an inner side wall extending transversely from said ring-shaped wall portion to said top panel of said housing, said light cover and said housing together being configured to define a ring chamber disposed between said inner side wall and said outer side wall, said light cover including translucent material to permit passage of light through said light cover;
a plurality of LED lights being attached to said housing, said LED lights being disposed within said ring chamber;
a smoke detector unit being detachably mounted on said top panel of said housing, said smoke detector unit being surrounded by said inner side wall of light cover;
a cover plate being attached to said smoke detector unit, said cover plate being configured to extend outwardly toward and parallel to said ring-shaped wall portion of said light cover to obscure said smoke detector unit from view;
a plurality of apertures being disposed in each of said outer side wall of said housing and said inner side wall of said light cover, said apertures being configured to permit passage of smoke through said housing and said light cover to said smoke detector unit.

2. The illuminated smoke detector assembly of claim 1, wherein said apertures include top panel apertures disposed in said top panel of said housing.

3. The illuminated smoke detector assembly of claim 1, wherein said top panel includes a central mounting portion configured to be attached to a surface, said top panel includes a perimeter portion about said central mounting portion, said top panel apertures are disposed in said perimeter portion, said central mounting portion is raised with respect to said perimeter portion such that there is space between said perimeter portion and the surface to which said central mounting portion is attached to permit flow of smoke through said space to said top panel apertures.

4. The illuminated smoke detector assembly of claim 3, wherein said top panel apertures are disposed in a ring about said central mounting portion between said smoke detector unit and said inner side wall of said light cover.

5. The illuminated smoke detector assembly of claim 1, wherein said smoke detector unit includes a base portion detachably connected to said top panel of said housing and a detector portion detachably connected to said base portion.

6. The illuminated smoke detector assembly of claim 5, wherein said cover plate is attached to said detector portion of said smoke detector unit.

7. The illuminated smoke detector assembly of claim 1, wherein said apertures include outer side wall apertures disposed in said outer side wall of said housing.

8. The illuminated smoke detector assembly of claim 1, wherein said apertures include inner side wall apertures disposed in said inner side wall of said light cover.

9. The illuminated smoke detector assembly of claim 1, wherein each of said housing and said cover plate including opaque material configured to block passage of light.

10. The illuminated smoke detector assembly of claim 1, wherein said smoke detector unit is configured to also detect carbon monoxide.

11. The illuminated smoke detector assembly of claim 1, wherein further comprising a control unit being connected to said LED lights to control operation thereof.

12. The illuminated smoke detector assembly of claim 11, wherein said control unit is a wireless receiver disposed in said ring chamber, and said illuminated smoke detector assembly further comprises a remote control configured to communicate with said control unit to control operation of said LED lights.

13. The illuminated smoke detector assembly of claim 1, wherein:
said apertures include top panel apertures disposed in said top panel of said housing.
said top panel includes a central mounting portion configured to be attached to a surface, said top panel includes a perimeter portion about said central mounting portion, said top panel apertures are disposed in said perimeter portion, said central mounting portion is raised with respect to said perimeter portion such that there is space between said perimeter portion and the surface to which said central mounting portion is attached to permit flow of smoke through said space to said top panel apertures;
said top panel apertures are disposed in a ring about said central mounting portion between said smoke detector unit and said inner side wall of said light cover;
said smoke detector unit includes a base portion detachably connected to said top panel of said housing and a detector portion detachably connected to said base portion;
said cover plate is attached to said detector portion of said smoke detector unit;
said apertures include outer side wall apertures disposed in said outer side wall of said housing;
said apertures include inner side wall apertures disposed in said inner side wall of said light cover;
each of said housing and said cover plate including opaque material configured to block passage of light;
said smoke detector unit is configured to also detect carbon monoxide;
the illuminated smoke detector assembly further comprises a control unit being connected to said LED lights to control operation thereof; and
said control unit is a wireless receiver disposed in said ring chamber, and said illuminated smoke detector assembly further comprises a remote control configured to communicate with said control unit to control operation of said LED lights.

14. An illuminated smoke detector assembly comprising:
a housing including a rear panel and a perimeter wall extending perpendicularly from said rear panel, said perimeter wall including a lip edge defining an opening in said housing;
a light cover detachably connected to said housing, said light cover being a panel extending perpendicularly from said perimeter wall at said lip edge of said perimeter wall, said light cover and said housing together being configured to define a rectangular chamber, said light cover being smaller than said opening in said housing to create a gap between said light cover and said lip edge to permit passage of smoke into said rectangular chamber, said light cover including translucent material to permit passage of light through said light cover;
a plurality of LED lights being attached to said housing, said LED lights being disposed within said rectangular chamber; and
a smoke detector unit being detachably mounted to said housing within said rectangular chamber.

15. The illuminated smoke detector assembly of claim 14, wherein:
said perimeter wall including a top wall portion, a bottom wall portion, and two lateral wall portions connecting said top wall portion and said bottom wall portion;
said smoke detector unit includes a cover frame configured to house components of said smoke detector unit, said cover frame is attached to said top wall portion; and
said gap is disposed between said light cover and said lip edge at said top wall portion, said cover frame is configured and disposed to block said gap, said cover frame includes a plurality of apertures adjacent said gap to permit passage of smoke through said cover frame.

16. The illuminated smoke detector assembly of claim 15, wherein said light cover includes instructional information to assist in exiting an area of a building in an emergency.

17. The illuminated smoke detector assembly of claim 16, wherein said smoke detector unit is configured to also detect carbon monoxide.

18. An illuminated smoke detector assembly comprising:
a housing including a base plate and a cover detachably connected to said base plate, said cover being dome-shaped, said base plate being configured to be attached to a ceiling;
a smoke detector unit being detachably mounted on said base plate within said cover;
a plurality of apertures being disposed in said cover of said housing, said apertures being configured to permit passage of smoke through said cover to said smoke detector unit; and
a chandelier being connected to and configured to be suspended from said cover of said housing.

19. The illuminated smoke detector assembly of claim 18, wherein:
said chandelier includes a central support assembly connected to said cover, a hub disposed at a free end of said central support assembly, a plurality of arms extending from said hub, and a plurality of lighting fixtures connected to said arms; and
the illuminated smoke detector assembly further comprises a battery and wiring connecting said battery to said smoke detector unit, said battery is disposed within said hub, said wiring extends along said central support assembly from said hub to said housing.

20. The illuminated smoke detector assembly of claim 19, wherein said smoke detector unit is configured to also detect carbon monoxide.
